(19) United States
(12) Patent Application Publication
(10) Pub. No.: US 2004/0087031 A1
(43) Pub. Date: May 6, 2004

(54) PH measurement system

(76) Inventor: Richard K. Simon JR., College Station, TX (US)

Correspondence Address: Stephen D. Dellett, TROP, PRUNER & HU, P.C., Suite 100, 8554 Katy Freeway, Houston, TX, 77024-1841, US

(21) Appl. No.: 10/288,533

(22) Filed: Nov. 5, 2002

(51) Int. Cl. G01N 33/00; G01F 1/64; G01N 27/26; G01N 31/16

(52) U.S. Cl. 436/100; 205/787.5; 436/163; 436/43; 436/50; 422/67; 422/68.1; 422/75; 422/82.12

(57) ABSTRACT

A pH measurement system automatically measures the pH of samples in purge and trap sample concentrators, autosamplers or other sample delivery devices. The pH measurement system includes a pH sensor that is positioned in a pH sample reservoir. The pH sample reservoir may be automatically filled with sample from a sparge vessel, calibrated using liquid having known pH, or rinsed. A controller enables and disables the valves and pump to transmit the liquid to the pH sample reservoir.

### BACKGROUND

The present invention relates generally to measurement of pH of samples that are analyzed using gas chromatography techniques. More specifically, the present invention relates to pH measurement of samples that are concentrated and tested for volatile organic compounds using gas chromatography techniques.

Sample concentration is used in purge and trap, headspace and thermal desorption gas chromatography (“GC”) analysis. Purge and trap sample concentrators are used to extract volatile analytes from a sample and concentrate them onto or into a sorbent trap. Purge and trap sample concentrators purge a known aliquot of a sample with a controlled flow rate of gas, with the aliquot being held in a sparge vessel. The sparge vessel typically is a tube designed to hold a liquid sample into which a gas such as helium or nitrogen is introduced. For example, a gas may be introduced into the sparge vessel through a needle. Or the sparge vessel may be a “U” shaped tube with a frit in one side to enhance bubble production from the gas, which enhances the rate of removal of volatile species from the aliquot of sample. When sparge gas is introduced into a sparge vessel, the volatile components may pass from the sparging vessel to a sorbent trap. After the volatile analytes and a relatively small amount of water, i.e., less than 0.1 mL, are removed from the sparge vessel to the trap, subsequent desorption and transfer of the volatile components from the trap to a gas chromatograph provides for separation and quantitative analysis of volatile analytes.

Autosamplers are automated sample delivery devices that deliver samples into a purge and trap system, or into one or more other devices that collect, analyze and test the sample. For example, autosamplers may include apparatus for sequentially removing a sample or other liquid from one or multiple vessels, and moving the sample or liquid into a sparge vessel in a purge and trap system. Autosamplers also may include a loop having a known volume, or other volumetric measuring device, to measure the sample before the sample is provided to the purge and trap system. Autosamplers also may rinse the loop or other components with water or other rinse solution between each sample.

There are several reasons why it may be desirable to measure the pH of samples in purge and trap sample concentrators, autosamplers and/or other automated sample delivery devices. A sample may be subject to pre-acidification to enhance the stability of analytes. For example, the Environmental Protection Agency (EPA) has specified that samples should be acidified as preservation technique for the volatile organic compounds that may be in the sample. Acidification may help prevent or delay biodegradation of the compounds between the sampling and analysis. For some of the compounds being analyzed by these methods, it has been suggested and reported that acidification may convert some of the compounds of interest into other compounds. As a result, some of the compounds of interest may be under-reported. These and other concerns about sample acidification may encourage the consideration and/or use of alkaline preservation techniques instead of acidification.

Additionally, some methodologies require verification of the pH of the sample. In the past, however, measuring the pH of a sample was not integrated or automated with a purge and trap sample concentrator, autosampler, or other sample delivery device, but instead involved unscrewing the sample cap, and then manually testing the sample with litmus paper, pH probes, or pH indicating dyes (colorimetric). As a result, pH measurement involved risks of misreading, damage to sample integrity, or improper recording.

Similarly, pH meters that are not integrated with purge and trap sample concentrators, autosamplers, or other sample deliver devices also require manual intervention for calibration and individual sample readings. The pH sensor or probe must be manually placed into vessels containing the sample or buffer solution(s). There also can be problems tracking, maintaining and ensuring the accuracy of data that depends at least in part on manual recordkeeping of pH measurements.

### BRIEF DESCRIPTION OF THE DRAWINGS

FIG. 1 is a schematic of a pH measurement system according to one embodiment of the invention.

FIG. 2 is a block diagram of the operation of a pH measurement system according to one embodiment of the invention.

FIG. 3 is a block diagram of specific operations of a pH measurement system according to one embodiment.

FIG. 4 is a block diagram of a pH measurement system according to another embodiment of the invention.

### DETAILED DESCRIPTION

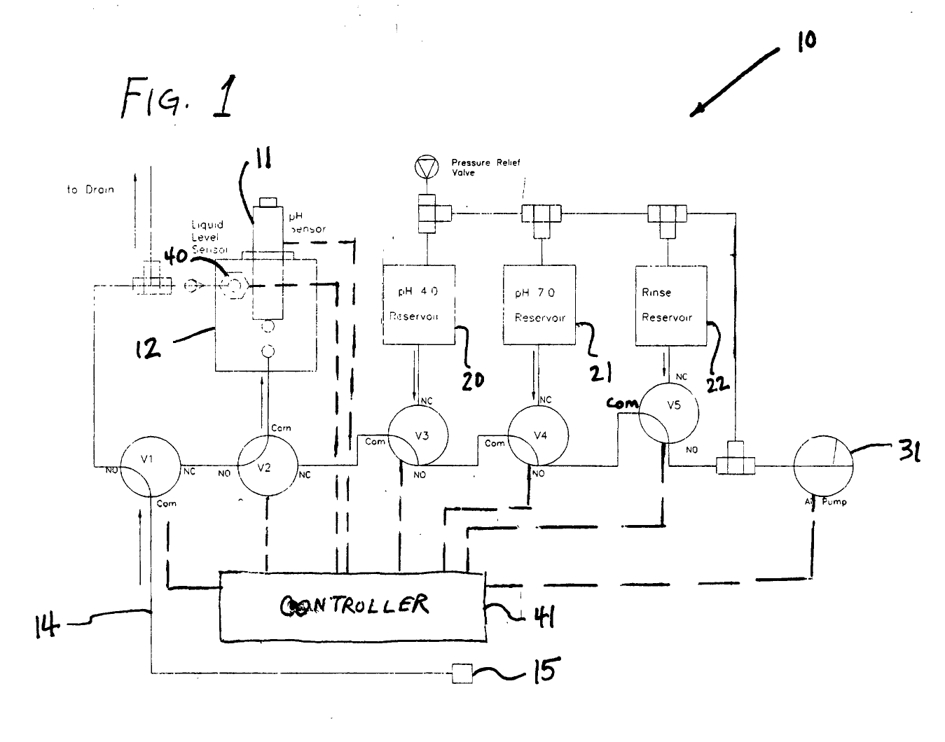

As shown in FIG. 1, pH measurement system 10 includes pH sensor 11 that may be positioned in pH sample reservoir 12. In accordance with the present invention, any type of pH sensor may be used. In one embodiment, a general purpose single junction pH sensor is used, such as model number HODK-0011 made by Analytical Sensors, Inc. The pH sensor may include both a sensing half cell and a reference half cell (with silver/silver chloride gel or silver/silver chloride solution). Other pH sensors that may be used with the invention include double junction, low alkali, high temperature, pH-FET sensors, and colorimetric sensors.

In one embodiment of the invention, the pH sensor may be allowed to stabilize prior to measuring the pH of the sample or other liquid in the sample reservoir. The length of time to stabilize and measure the pH depends on a number of factors including but not limited to the age of the pH sensor, the measurement frequency, and the acceptable margin of error or uncertainty in pH readings. In one embodiment, the pH sensor measures the pH of the sample or other liquid in the pH sample reservoir for one minute, at a frequency of 20 Hertz, to determine the pH within plus or minus 0.01 pH unit per measurement. The time period, frequency, and error margin may be greater or less in other embodiments of the invention. For example, in one embodiment of the invention, the time period may be 30 seconds, the frequency anywhere from 1 Hz to 100 Hz, and the error margin may be 0.05 to 0.1 pH unit per measurement.

A temperature sensor also may be included in one embodiment of the invention, to measure the temperature of the sample or other liquid in the pH sample reservoir. For example, the temperature sensor may be built into the pH sensor. Examples of temperatures sensors that may be used with the invention include platinum resistance thermometers, thermistors, or thermocouple junctions.

The response of the pH sensor is temperature dependent, and the temperature sensor allows the pH measurement to be corrected. For example, the temperature of a sample in the pH sample reservoir may be elevated above ambient because the sample may be heated in the sparge vessel. While the sparge vessel is purged by gas and the volatile analytes are transferred to a sorbent trap, the sparge vessel may be heated by an immersion heater, a radiative heater or a conventional blanket heater. As a result, the sample in the pH sample reservoir may be above ambient temperature.

The pH for an aqueous solution may be calculated with the following formula:

pH=pHstd+(E−Estd)×F/(R×T×1n(10)) or

pH=pHstd+(E−Estd)/(0.19841×T)

where

pH is the pH of the sample,

pHstd is the pH of the standard,

E is the measured cell potential of the sample (in mV),

Estd is the measured cell potential of the standard (in mV),

F is the Faraday constant (96487 Coul/mole),

R is the gas constant (8.3143 J/mole-degree K),

T is the temperature of the sample in degrees Kelvin.

In one embodiment of the invention, the measured voltage E of the sample or other liquid may be amplified and converted into a digital form using an analog to digital converter (ADC). In converting the measured voltage to a digital form, there is an offset and gain. The offset may correspond to the digital value for an input voltage of 0 volts, preferably at the isopotential point (i.e., pH of 7.00). The gain is the slope of a plot of the voltage input versus the reported digital output. The slope may be determined using a sample buffer solution having a known pH.

The pH sample reservoir 12 may be a vessel fabricated from any material that can withstand the acidic environment of the sample or other liquid. For example, the pH sample reservoir may be made from Teflon, Kynar, polysulfone, polypropylene, or any other material compatible with acidic, neutral or basic aqueous solutions. In one embodiment, the pH sample reservoir is a Kynar cell. Preferably, the pH sample reservoir is sized to permit the sample or other liquid to cover the pH electrode submersed therein, while at the same time having sufficient space for the entrained gas. Thus, the pH sample reservoir may be of sufficient size and dimension so that bubbles rising from the sample or other liquid partially fill the upper volumetric region of the pH sample reservoir and do not adversely affect the pH measurement. In one embodiment, the volume of the pH reservoir allows effective transfer of small aliquots (i.e., 5 mL) into the pH sample reservoir without bubbles adversely affecting the pH measurement.

The pH sample reservoir may be connected through conduit 13 to sparge vessel 15 or another sample delivery device such as an autosampler. In one embodiment, the pH sample reservoir also may be connected through conduits to one or more reservoirs for holding buffer or rinse solutions. The conduits may be passages in and/or through a manifold or other body, or may be tubing such as ⅛ inch OD, 1/16 inch ID Teflon tubing. Seals at the conduit junctions, and the seal between the pH sensor and the pH sample reservoir, may be compatible with the sample solution and provide leak tight seals, such as a compressed O-ring type seals.

In one embodiment of the invention, the pH measurement system may be calibrated, rinsed, and prepared for pH measurement before, after and/or between the introduction of each sample or other liquid into the pH sample reservoir. This may be done by introducing one or more buffer solutions, rinse solutions, air or other gas into the pH sample reservoir before, after and/or between the pH measurement.

For example, in one embodiment of the invention, air or other gas such as nitrogen or helium may be used to purge the system prior to loading each sample or other liquid into the pH sample reservoir. Pre-purging the pH sample reservoir with gas helps ensure that the liquid level sensor detects the arrival of a new sample or liquid instead of the previous one. Air or other gas also may be used to remove the sample or other liquid from the pH sample reservoir.

In one embodiment, the invention provides the capability of calibration of the pH sensor. This embodiment may include at least one reservoir or vessel for holding liquid buffer solution having a known pH for calibrating the pH sensor. For example, in the embodiment of FIG. 1, reservoir 20 contains pH 4.0 buffer solution, and reservoir 21 contains pH 7.0 buffer solution.

In one embodiment, the invention also provides the capability of rinsing the pH sample reservoir. This embodiment may include a reservoir or vessel for holding a rinse solution such as de-ionized water. As shown in FIG. 1, reservoir 22 contains a rinse solution, which may be deionized water.

In one embodiment of the invention, a pump may be used to move the buffer solution(s), rinse solution, and/or sample into and out from the pH sample reservoir. For example, the pump may be an air pump or vacuum pump. In the embodiment of FIG. 1, for example, air pump 31 moves the liquid from buffer or rinse vessels into the pH sample reservoir. An air pump may provide sufficient pressurization and an acceptable air flow rate to move the liquid from the buffer or rinse solution reservoirs into the pH sample reservoir in an acceptable time period. In one embodiment of the invention, a diaphragm pump may be used, such as the Model BP-202-1 produced by Binaca Products of Temecula, Calif. This pump provides up to 3 L/min air flow with a maximum head pressure of 6 psig. Pumps other than air or vacuum pumps, such as metering pumps, also may be used in the present invention.

In one embodiment of the invention, liquid level sensor 40 may be provided to detect a liquid volume or liquid level in the pH sample reservoir. For example, in the embodiment of FIG. 1, the specified liquid volume, which is based on the size of the pH sample reservoir, may be 3.7 mL. One type of liquid level sensor that may be used in the invention is the model LLE105000 made by Honeywell. This sensor operates on the principle of refractive index difference between air and water, and is not affected by ionic strength of the solution. Other liquid level sensors may be used to detect the liquid volume in the pH sample reservoir, such as conductivity sensors or sensors that capacitively or inductively sense the presence of the sample. A liquid level sensor may be inside or outside the pH sample reservoir. Alternatively, a flow detector may be used instead of a liquid level sensor to detect the volume of liquid in the pH sample reservoir.

Flow of the sample, buffer, and/or rinse solutions into the pH sample reservoir may be controlled by one or more valves. In one embodiment, the valves are isolation valves such as ASCO/ANGAR 3-way isolation valves (Angar Scientific C., Cedar Knolls, N.J.). The valves may be manifold style isolation valves or other valves made from inert materials capable of withstanding acidic solutions.

In the embodiment of FIG. 1, one or more of valves V1-V5 are selectively opened and closed to fill or empty the pH sample reservoir with the sample, the buffer solution(s) or rinse solution. Each of valves V1-V5 has a normally open position and a normally closed position. In the normally open position, the valve is open between the common and the NO port. When the valve is enabled, the valve is switched from the normally open position to the normally closed position where the valve is open between the common and NC port.

In one embodiment of the invention, controller 41 may be included in the pH measurement system. The term “controller” includes any microcontroller or other device on which one or more components rely for access to a computer subsystem, and also may include a processor, microprocessor or computer, and associated software, firmware and/or hardware. The functions of the controller may include but are not limited to data acquisition (i.e., pH, temperature data), data recording in machine readable storage medium such as a disk or memory, selection of the liquid or sample source, monitoring the pH sensor, clocking functions, interrupt sensing, control of valves, relays and pumps, monitoring the liquid level sensor or other sensors, and communicating with other processors, computers or devices. One example of a suitable controller that may be used in one embodiment of the present invention is a Microchip PIC16F876 that includes built in serial ports, I/O ports, and flash memory. Another example is a CIO-DAS8 board sold by Computer Boards, Inc. which may be installed into a personal computer. The CIO-DAS8 board has an 8254 counter timer chip and an 8255 digital I/O chip. The 8254 chip may be configured so that its counters are used to establish a waveform at the desired acquisition frequency for clocked analog to digital conversions. The CIO-DAS8 board may be hardwired such that the PC clock is divided down by counter 2 and the output from counter 2 is divided down by counter 1. The output from counter 1 is connected to the interrupt port. In this embodiment, pin 6 connects to pin 4, and pin 5 connects to pin 24 on the DAS8 connector, and the system timer clock is 4.0 Mhz. The controller or processor may be programmed using software languages such as C or C++ or other code to enable control circuitry to perform one or more of the above functions relating to pH measurement according to desired and repeatable sequences. The controller may be connected to one or more components of the pH measurement system including pH sensor 11, temperature sensor 43, valves V1-V5, pump 31, other sensors such as pressure sensor 42, and also may be linked or connected to controller 44 for the purge and trap sampler concentrator and/or autosampler, and to operating system 45 including memory or machine readable storage device(s) 46. Machine readable storage includes but is not limited to any computer memory or storage device including disk or tape storage, as well as storage in remote locations.

In one embodiment of the invention, the controller may have a register to enable control circuitry for each of the valves, pump, liquid level sensor, and/or other sensors. For example, each bit on the register may be associated with valves V1-V5, a relay for the pump, and the liquid level sensor.

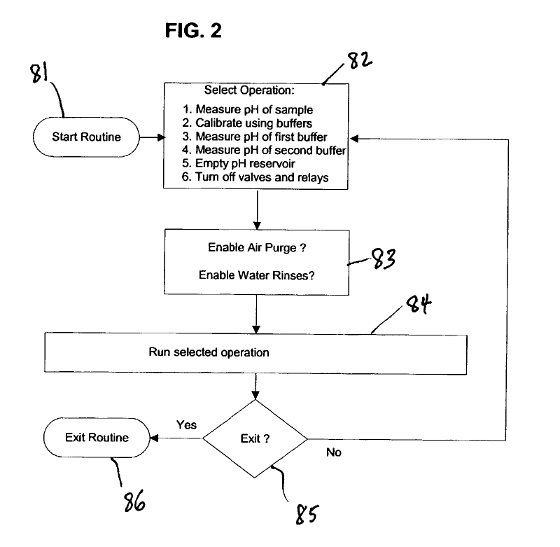

FIG. 2 is a block diagram showing the operation of the pH measurement system according to one embodiment of the invention. In this embodiment, the pH measurement system may run the following operations: (1) Measure the pH of the sample from the purge and trap sample concentrator or sample delivery device; (2) Calibrate the pH sensor with two buffer solutions, each having known pH; (3) Measure the pH of a first buffer solution having a known pH; (4) Measure the pH of a second buffer solution having a known pH; (5) Empty the pH sample reservoir; or (6) Turn off all valves and relays.

As shown in FIG. 2, block 81 is labeled Start Routine. In block 82, the user may specify the desired operation of the system. In block 83, the user may select whether or not to enable air purge and/or water rinse before and between each run. In block 84, the selected operation is run, which will be described in more detail below. In block 85, there is a test to determine if the user seeks to exit the routine in block 86. If not, another operation may be selected again in block 82.

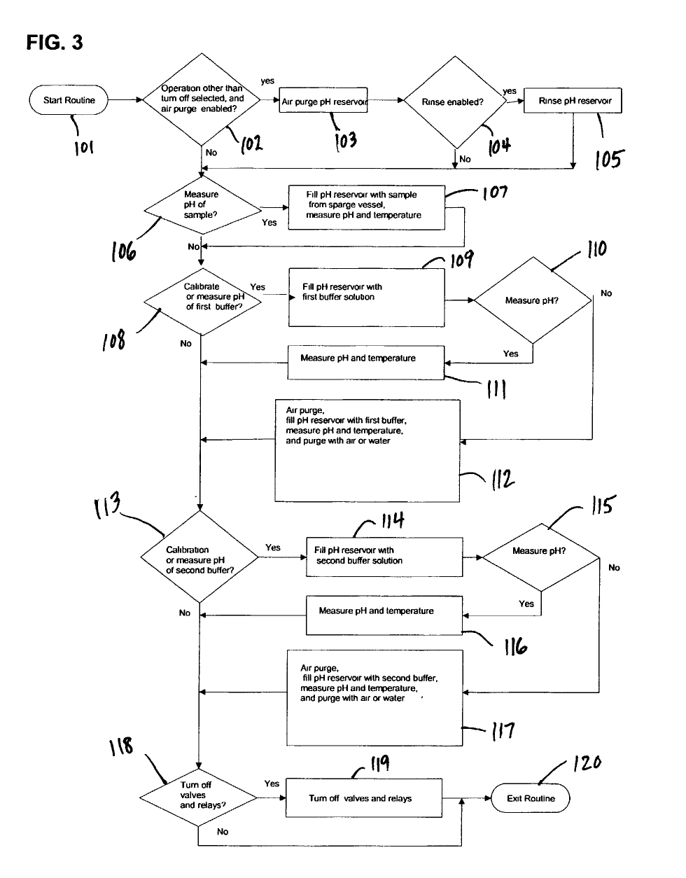

FIG. 3 is a detailed block diagram of the specified operations identified in block 84 in FIG. 2, according to one embodiment of the invention. The routine starts in block 101. Block 102 tests if one of the above-listed operations (other than Turn Off all valves and relays) was selected, and if air purge was enabled. If Yes, in block 103 the controller enables a relay to activate the air pump and enables valve V2 to purge the pH sample reservoir with air for a desired time period, e.g., 30 seconds. In block 104, the controller tests if water rinse is enabled and if Yes, in block 105 enables a relay to activate the air pump, and enable valves V2 and V5 so that rinse solution flows into the pH sample reservoir. When the liquid level sensor in the pH sample reservoir detects a full volume, the controller deactivates the air pump, returns valve V5 to its normally closed position. The rinse solution may remain in the pH sample reservoir for a desired time period, and may be drained by opening valve V2 and activating the air pump.

In one embodiment of the invention, in block 106, the controller tests if measuring the pH of the purge and trap sample was selected. If Yes, in block 107, the controller enables valve V1 so that the sample from the sparge vessel enters the pH sample reservoir. When the liquid level sensor detects a full volume, the controller returns valve V1 to its normally closed position and the pH and temperature of the sample is measured for a specified time period. The sample may be drained from the pH sample reservoir by opening valve V2 and activating the air pump.

In one embodiment of the invention, in block 108, the controller tests if calibrating the pH sensor, or measuring the pH of a first buffer solution was selected. For example, the first buffer solution may have a pH of 7.0. If calibration or measuring the first buffer pH was selected, in block 109, the controller enables a relay to activate the air pump and enable valves V2 and V4 so that the first buffer solution enters the pH sample reservoir. When the liquid level sensor detects a full volume, the controller deactivates the air pump and returns valves V2 and V4 to their normally closed positions. In block 110, the system tests if calibration or pH measurement was selected. In block 111, the pH and temperature of the first buffer solution are measured for a specified time period. Optionally, as shown in the embodiment of FIG. 3, if calibration was selected, the first buffer solution may be drained by opening valve V2 and activating the air pump, and the pH sample reservoir may be air purged, as shown in block 112. After air purge, the pH sample reservoir is filled a second time with the first buffer solution. The pH and temperature of the first buffer solution then may be measured for specified time period. The first buffer solution may be drained from the pH sample reservoir. Then, if so enabled, the sample reservoir may be purged with air and/or rinsed with water.

In one embodiment of the invention, in block 113, the controller tests if calibrating the pH sensor, or measuring the pH of a second buffer solution was selected. The second buffer solution, for example, may have a pH of 4.0. If calibration or measuring the second buffer pH was selected, in block 114, the controller enables a relay to activate the air pump and enable valves V2 and V3 so that the second buffer solution enters the pH sample reservoir. When the liquid level sensor detects a full volume, the controller deactivates the air pump and returns valves V2 and V3 to their normally closed positions. In block 115, the controller tests if calibration or pH measurement was selected. In block 116, the pH and temperature of the second buffer solution are measured for a specified time period. Optionally, if calibration was selected, the second buffer solution may be drained from the sample and, in block 117, the pH sample reservoir may be air purged and then filled a second time with the second buffer solution. The pH and temperature of the second buffer solution are measured, and the pH sample reservoir then may be purged with air and/or water if so enabled.

In one embodiment of the invention, in block 118, the controller tests if the operation Turn off all valves and relays was selected. If Yes, in block 119, the controller turns off all valves and relays. Block 120 then exits the routine.

In one embodiment of the invention, the user may measure the pH of the same sample or buffer in the pH sample reservoir more than once. If desired, air purges and/or water rinses may precede or take place between each of the repeat measurements and/or samples, time delays may be programmed between each repeat measurement, and the pH sample reservoir may be drained between each measurement.

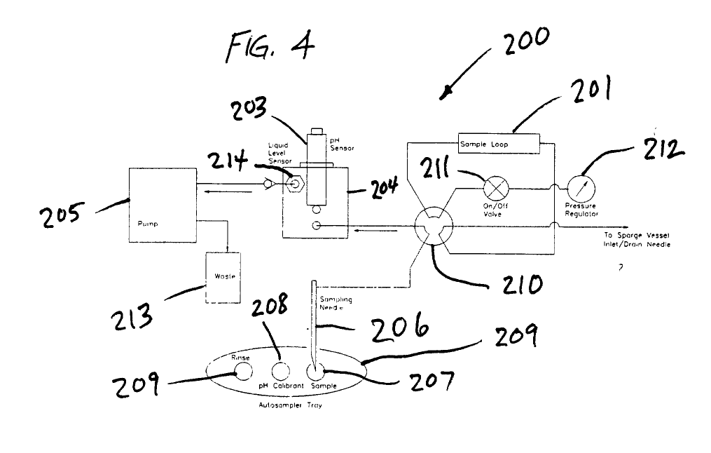

FIG. 4 shows an embodiment of the invention in which pH measurement system 200 is connected to an autosampler, such as the OI Corporation model 4551a autosampler. In a typical autosampler, a specified volume of sample or other liquid from one or each of multiple vessels may be measured using sample loop 201 or a similar measuring apparatus, before the sample is provided to sparge vessel 202. Typically, measurement with a sample loop results in some excess sample. During or after measuring, excess sample or other liquid may be sent to a drain or waste container. In this embodiment, however, some of the sample or other liquid may be directed through a conduit to the pH sample reservoir instead of the drain or waste container. For example, excess sample from measurement in the sample loop may be directed to the pH sample reservoir. This may be done by inserting the pH measurement system between sample loop 201 and waste container 213. The pH sensor 203 may measure the pH of sample liquid in pH sample reservoir 204 in a manner similar to the other embodiments described previously.

In the embodiment of FIG. 4, air pump 205 and sampling needle 206 may remove liquid from sample vessel 207, shown in autosampler tray 209, through valve 210 into a sample loop to measure a specified volume. Valve 210, which may be a six port valve, may be connected and configured to direct excess liquid from the sampling needle and/or sample loop through conduits to the pH sample reservoir. In one embodiment, pH calibrant vessel 208 and/or rinse vessel 209 may be provided to store solution having known pH or rinse solution for the pH sample reservoir. In this embodiment, pH calibration standards may be included in one or more vessels in the autosampler. Also shown in FIG. 4 is on-off valve 211 and pressure regulator 212, and liquid level sensor 214.

Once the autosampler provides a measured sample to the sparge vessel, the pH of the sample may be measured prior to, during, or after purging the analytes from the sparge vessel. One advantage of the embodiment of the invention is that the sample for pH measurement has reduced entrained air bubbles. In one embodiment of the invention, liquid samples also may be directly and/or manually injected into the pH sample reservoir. For example, a fitting may provide access to a conduit into the pH sample reservoir.

In one embodiment of the invention, testing, configuration and/or diagnostic functions also may be provided in a pH measurement system. These functions may be accessed through a menu or other means. For example, testing or diagnostic functions may involve testing the valves, air pump and liquid level detector by filling the pH sample reservoir one or more times with rinse solution, buffers or samples from the sample concentrator. The pH sample reservoir may then be purged or rinsed between tests. Another example is calibrating the temperature sensor by connecting a known resistance (i.e., 100 ohms) to the temperature sensor to calculate the gain and offset with a two point calibration curve, i.e., one point on the curve for 0 degrees C. and another point representing 100 degrees C. Other examples include testing the liquid level sensor, testing the toggle valves, and testing the pH sensor and temperature sensor and conversion of analog to digital data. Each of the above testing functions may be programmed using software.

Other examples of configuration functions are setting acquisition frequencies, listing current user settings, opening and writing configuration and diagnostic information to configuration files or Comma Separated Variable Files, such as pH and temperature calibrations (i.e., gain and offset), and enabling logging flags. When logging flags are enabled, print statements may permit examination of calculations and intermediate steps in fine detail. Additionally, statistical information concerning the pH and temperature data may be collected over a specified number of readings, such as 20 or 100, calculated and stored, including averages, standard deviations, minimums and maximums.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. A pH measurement system comprising:
a pH sample reservoir;
a pH sensor positioned in the pH sample reservoir;
a sparge vessel and a conduit from the sparge vessel to the pH sample reservoir; and
at least one valve in the conduit to control flow between the sparge vessel and the pH sample reservoir.

2. The pH measurement system of claim 1 further comprising a controller coupled to the pH sensor and the at least one valve.

3. The pH measurement system of claim 1 further comprising a temperature sensor positioned in the pH sample reservoir.

4. The pH measurement system of claim 1 further comprising a liquid level sensor to sense the liquid level in the pH sample reservoir.

5. The pH measurement system of claim 1 further comprising at least one reservoir to hold a liquid having known pH and at least one valve to control flow of the liquid having known pH into the pH sample reservoir.

6. The pH measurement system of claim 1 further comprising a reservoir to hold a rinse liquid and at least one valve to control flow of the rinse liquid into the pH sample reservoir.

7. The pH measurement system of claim 1 further comprising a plurality of valves to control liquid flow into the pH sample reservoir, and a controller connected to the valves to control the opening and closing of the valves.

8. A pH measurement system comprising:
a pH sample reservoir;
a pH sensor positioned in the pH sample reservoir;
an automated sample delivery device and a conduit between the automated sample delivery device and the pH sample reservoir; and
at least one valve in the conduit to control flow between the automated sample delivery device and the pH sample reservoir.

9. The pH measurement system of claim 8 further comprising a controller coupled to the pH sensor and the at least one valve.

10. The pH measurement system of claim 8 further comprising a temperature sensor positioned in the pH sample reservoir.

11. The pH measurement system of claim 8 further comprising a liquid level sensor to sense the liquid level in the pH sample reservoir.

12. The pH measurement system of claim 8 further comprising at least one reservoir to hold a liquid having known pH and at least one valve to control flow of the liquid having known pH into the pH sample reservoir.

13. The pH measurement system of claim 8 further comprising a reservoir to hold a rinse liquid and at least one valve to control flow of the rinse liquid into the pH sample reservoir.

14. The pH measurement system of claim 8 further comprising a plurality of valves to control liquid flow into the pH sample reservoir, and a controller connected to the valves to control the opening and closing of the valves.

15. A method comprising:
transmitting a liquid sample through a conduit from a sparge vessel to a reservoir having a pH sensor therein;
measuring the pH of the liquid sample in the reservoir; and
emptying the liquid sample through a conduit from the reservoir to a drain.

16. The method of claim 15, further comprising measuring the temperature of the liquid sample in the reservoir.

17. The method of claim 15, further comprising transmitting a rinse liquid through a conduit from a vessel to the reservoir having a pH sensor therein.

18. The method of claim 15, further comprising transmitting a liquid having a known pH through a conduit from a vessel to the reservoir having a pH sensor therein.

19. The method of claim 15, further comprising recording the pH of the liquid sample in a machine readable storage medium.

20. The method of claim 15 further comprising calibrating the pH sensor by transmitting at plurality of liquids having known pH through conduits to the reservoir having a pH sensor therein, and measuring the pH of each of the plurality of liquids.

21. The method of claim 15 further comprising detecting the liquid level in the reservoir.

22. The method of claim 15 further comprising moving gas into the reservoir.

23. An apparatus for measuring pH of liquid comprising:
a first vessel to hold a liquid and having a pH sensor positioned therein to obtain pH measurements of the liquid;
a conduit between the first vessel and a plurality of other vessels, at least one of the other vessels being a sparge vessel;
a plurality of valves to control the flow of the liquid from each of the plurality of other vessels into the first vessel; and
a controller operably coupled to the plurality of valves and the pH sensor to automatically enable and disable the valves and receive pH measurements from the pH sensor.

24. The apparatus of claim 23 further comprising machine readable storage connected to the controller to store pH measurements received from the pH sensor.

25. The apparatus of claim 23 further comprising a liquid level detector connected to the controller to detect the liquid level in the first vessel.

26. The apparatus of claim 23 further comprising a temperature sensor connected to the controller to sense the temperature of the liquid in the first vessel.

27. The apparatus of claim 23 further comprising a pump connected to the controller to urge the flow of liquid from at least one of the plurality of other vessels into the first vessel.

28. The apparatus of claim 23 further comprising a gas source connected to the controller to provide gas through a conduit to the first vessel.

29. A method comprising:
purging analytes from a liquid sample in a sparge vessel to a sorbent trap;
transmitting the liquid sample from the sparge vessel through a conduit to a reservoir having a pH sensor therein;
measuring the pH of the liquid sample; and
recording the pH measurement in machine readable storage.

30. The method of claim 29 further comprising calibrating the pH sensor with at least one liquid having a known pH.

31. The method of claim 29 further comprising rinsing the reservoir with a rinse solution transmitted through a conduit into the reservoir.

32. A method comprising:
transmitting a liquid sample from a sample container into a container having a measured volume and an excess volume;
transmitting the measured volume of liquid sample into a sparge vessel;
transmitting the excess volume of liquid sample into a reservoir having a pH sensor; and
measuring the pH of the excess volume of liquid sample.

33. The method of claim 32 wherein the container is a sample loop.

34. The method of claim 32 further comprising flowing a gas through the sparge vessel to a sorbent trap.

35. The method of claim 32 further comprising recording the pH measurements in machine readable storage.
